(19) United States
(12) Patent Application Publication
(10) Pub. No.: US 2008/0085507 A1
(43) Pub. Date: Apr. 10, 2008

(54) SYSTEM FOR RAPID ANALYSIS OF GLYCATED PROTEINACEOUS SPECIES IN BIOLOGICAL SAMPLES

(75) Inventors: Thomas Stroud, Overland Park, KS (US); David Stalling, Lenexa, KS (US); Thomas Dobbs, Gladstone, KS (US); Jeffrey Wiseman, Auxvasse, MO (US)

Correspondence Address: LATHROP & GAGE LC, 4845 PEARL EAST CIRCLE, SUITE 300, BOULDER, CO, 80301, US

(73) Assignee: Affinex, LLC, Overland Park, KS (US)

(21) Appl. No.: 11/868,425

(22) Filed: Oct. 5, 2007

Related U.S. Application Data
(60) Provisional application No. 60/849,532, filed on Oct. 5, 2006.
(60) Provisional application No. 60/938,982, filed on May 18, 2007.

(51) Int. Cl. G01N 33/72; C12Q 1/00; G01N 33/487

(52) U.S. Cl. 435/4; 436/67; 436/87

(57) ABSTRACT

A device is described which provides an automated or manual means to perform chromatographic affinity-based A1c analyses of whole blood or hemolysates in less than 1 minute. Further, such applications are useful for the quantitation of glycated plasma proteins used in analysis of gestational diabetes. The device includes several modules integrated to accomplish processing and analysis of the blood sample. One such module includes a disposable liquid chromatographic column which may be rapidly packed and is easily assembled from readily available materials. Such columns and assemblies may be used in fluid chromatography applications requiring separations of complex materials for purposes of purification and/or quantitation of particular analytes. The columns may be used as stand-alone units or may be integrated into commercially available chromatography systems. The columns have a chemical composition and particle size that allow for low operational pressures.

The present application claims benefit of priority to U.S. Provisional patent application Ser. Nos. 60/849,532 filed Oct.5, 2006 and 60/938,982 filed May 18, 2007, both of which applications are hereby incorporated by reference to the same extent as though fully replicated herein.

### BACKGROUND

1. Field of Disclosure

The disclosure generally relates to medical diagnostic instrumentation that incorporates one or more chromatographic systems capable of performing chromatographic separations, and subsequent quantifications of different fractions of proteins in blood samples.

2. Description of Related Art

Diabetic patients suffer from abnormally high levels of sugar in their blood. Abnormally high blood sugar may result in irreversible damage to various organs or tissues. It is therefore critical that the blood sugar levels of a diabetic patient be closely monitored. Hemoglobin A1c (also known as HgbA1c or A1c) refers to a measure of the percentage of hemoglobin A1 that has been glycosylated. Measurement of A1c has been recommended by the American Diabetes Association (ADA) as an important tool for monitoring the health condition of diabetic patients.

Analysis of blood samples for A1c typically starts with a sample containing blood cells that may be drawn by such means as a finger prick or venipuncture. The sample may then be diluted in a lysing solution to expose proteins thereby creating a hemolysate. The hemolysate may be contained in a sealed tube (e.g. Hemogard™) which may either be individually transported for immediate injection or be transported to/from a sample storage rack and possibly identified by bar code reading or radio frequency identification technology (RFID) for subsequent injection into a chromatographic system consisting of a liquid high pressure pump, injector, chromatographic column, detector and some method of signal processing such as data integrator or the like.

Current systems often fail to meet a need for rapid automated or manual systems to process samples and deliver analytical results in a short period of time. Numerous clinical studies indicate that patients significantly benefit from rapid A1c test results. According to the ADA, American Medical Association (AMA), the Center for Disease Control (CDC) and other health-related organizations, this enables caregivers to more expeditiously adjust treatments and offer immediate face-to-face counseling for improved medical outcomes.

Facilities for treating diabetic patients may or may not have operators and laboratory space certified to accommodate more complex analytical devices. Therefore, it can be preferable to have an analytical device that does not require these certified resources to reliably and rapidly deliver test results of sufficient accuracy to support more effective rapid treatment. Furthermore, facilities for treating diabetic patients may experience wide fluctuations in the volume of diabetic patients requiring testing within a given timeframe. Therefore, it can be preferable to have analytical devices capable of handling higher patient volumes within a given timeframe in order to deliver the more effective rapid treatment without undue queuing penalty.

As reported by U.S. Pat. No. 5,843,788, the analysis of blood samples for A1c Hemoglobin uses a known quantity of whole blood. The red blood cells in the sample are lysed to release the hemoglobin for subsequent analysis. These processed blood samples with the red blood cells lysed are commonly referred to as hemolysates. Ensuring that the hemolysate concentration falls within a narrow range may enhance the accuracy of the A1c measurement. This is useful because improper concentration of hemolysates resulting from either over-dilution of a strongly anemic patient's sample or under-dilution of blood sample from an individual with high hematocrit values may lead to inaccurate A1c readouts. In addition, a sample with excessively high levels of protein may overload the capacity of the separation column or cartridge and lead to aberrant results. In an automated system, it is beneficial to control or adjust the hemoglobin concentrations of the blood samples to fall within a pre-determined optimal range in order to achieve accurate measurements.

Red blood cells often contain proteins similar to but different from the glycated proteins measured to determine the patient's A1c level. Care must be taken to differentiate these blood variants from A1c in order to produce reliable results of sufficient accuracy for effective treatment.

Moreover, certain proteins are glycated and transported via the serum portion of the blood. In case of pregnancies, there are often transient diabetic conditions which require medical monitoring. Such cases, also known as gestational diabetes, may be detected or monitored by measurement of glycated plasma proteins as a method of determining elevated blood glucose that leads to glycation of plasma proteins.

### SUMMARY

The instrumentalities disclosed herein overcome the problems outlined above and advance the art by providing for the reliable rapid analysis of samples that may be contained in individualized tubes. An automated system is provided which is capable of rapidly measuring the A1c levels of a plurality of blood samples. The system may include a plurality of modules to accomplish processing and analysis of the blood samples. A module may contain a single device or a plurality of elements that may operate together to exert certain physical, chemical or biological effects on the samples. The physical processing of the samples may include, for example, relocating, mixing, rotating, spinning, sampling, heating, cooling, separating or filtering the samples. The chemical or biological effects may include, for example, chemical separation, modification, precipitation, macromolecule assembly and disassembly, attachment through chemical bonding or biological affinity.

The system may benefit from a modularized construction, which is more convenient to maintain or repair. System modules may obtain information from the samples at various steps throughout the process. For instance, a bar code scanning module may be used to read bar code information from the container, while an optical detector module emits light through the sample and reads the optical density of each sample. Yet other modules may communicate with other devices or the human operator. Certain functions or effects may require more than one module acting together. Conversely, one module may accomplish more than one function or have more than one effect on a sample.

Samples converted into a hemolysate by means of manual agitation or an automated hemolysis module may be presented to a fluidics module. The fluidics module controls flow of the mobile phase for the sample processing module and the chromatographic columns. The automated system may include one or more liquid chromatographic columns (or cartridges) to separate different hemoglobin fractions. A mobile phase pre-heater and a cartridge oven may be employed to maintain the temperature of the mobile phase and the separation cartridge.

The columns are preferably disposable which may be easily assembled from relatively inexpensive and readily available materials. Such columns and assemblies may be used in fluid chromatography applications requiring separations of complex materials for purposes of purification and/or quantitation of particular analytes. The columns may be used as stand-alone units or may be connected to commercially available chromatography systems. In one aspect, the columns may include one or more Luer type connectors to facilitate connections to other modules or devices.

An optical detector may be included to monitor the optical density of the effluent of the cartridge at 415 nM. Alternatively, in the case of plasma proteins, a wavelength suitable for analytes which are glycated is chosen, most preferably at 280 nM. A computer program records the chromatographic profile after the absorbance values of the detector's analogue output is digitized at 20 pts/sec or the like. The program provides data reduction to yield chromatographic baseline corrected area ratios for patient samples; these ratios are subsequently converted to A1c values via the system's current calibration curve. This curve is derived from the analysis of certified samples of known A1c value. Control charts for data quality are obtained by plotting analytical results for control samples against the range of their known values.

In one embodiment, a multi-fingered tube gripper module may be used to facilitate translocation of the sample tubes in the system. In addition to relocating the tubes from one location to another, the gripper module may be designed to allow tube rotation around the vertical axis of the tube such that tube identification may be read from the bar code on the tube. The bar codes are preferably affixed to the side of the tubes. The output shaft of a single reversible motor may provide both the gripping and rotating motions. The gripper/reader assembly may be attached to a robotic vial transport module to present individual tube to a septum piercing sampling needle so that samples may be picked up for further processing and analysis. The fluidics of the sampling needle may be presented to the fluidics module. In a similar manner, after analysis, the tubes may be presented to the gripper assembly for re-identification and return to the sample tray.

In another aspect, the system may include a tube-receiving and/or mixing module. Preferably, the tubes are presented to a tube-receiving and mixing module before being presented to a sampling needle. The tube receiving and mixing module may be designed to handle single or multiple tubes at a time. The main function of the tube-receiving module is to receive the tube from the gripper module after bar code information has been read. The mixing module serves to resuspend sedimented red blood cells to ensure that a uniform sample of whole blood is presented to the hemolysis and sample analysis modules. The tubes may be inverted vertically, swirled horizontally or rotated in any directions ranging from vertical to horizontal motion. The mixed blood samples may then be presented to the blood processing modules, which may include the Sample Intake Module, the Hemolysis Module, the Column or Disposable Cartridge Modules.

In another embodiment, valve rotors may be used to allow a single blood sample to be processed by different processing modules. Valve motors may also allow one device to be connected to other devices at different time during the process. For instance, a valve rotor containing multiple chambers may be employed to feed the blood samples into different blood processing modules. Briefly, blood may be input to one of the chambers in valve rotor with multiple chambers. The rotation of the valve rotor may move the blood from one chamber to another. Some chambers may be connected to different blood processing modules, while some chambers may not be connected to any blood processing modules. Thus, the rotation of the valve motor may render the blood sample accessible to different blood processing modules, which may include the Sample Intake Module, the Hemolysis Module, the Separation Module.

The system may also include an optional hemolysis module which may lyse the red blood cells in the samples. Use of the hemolysis module may not be required for all applications. For instance, samples of pre-prepared hemolysates may be directly analyzed after loading of the sample without going through the hemolysis module.

Under normal operation, a system operator may create a sample run map designating sample type (patient, calibration or control samples) and known data values (where appropriate). The program may then respond with appropriate actions to create System Calibration or quality control reports after calibration of the system. The operator may review and accept of the calibration curve results. The software may provide appropriate output and storage of calibration curves and/or data archival of patient data.

The instant disclosure also provides a methodology for optimizing the concentrations of hemoglobin such that the hemoglobin concentrations in different samples fall within a narrow range before passing by the detector. Under this scheme, the readouts from the system reflect the adjusted concentrations which may be converted by retrograde calculation to obtain the concentrations of the original samples.

### BRIEF DESCRIPTION OF THE DRAWINGS

FIG. 1 shows a gripper apparatus in an fully closed (A) or open (B) positions.

FIG. 2 shows the gripper assembly providing tube gripping and tube rotation functions, along with the motor.

FIG. 3 illustrates the means for adjusting the tension of the gripping arms.

FIG. 4 illustrates the gripping and rotation positions for tube transport/release (A) and tube rotation (B) for bar code identification.

FIG. 5 shows a single tube mixing station of the tube receiving and mixing module.

FIG. 6 illustrates schematically the components of the A1c Analysis System and the flow scheme of fluidics.

FIG. 7 shows an example of sample pipeline process employed by the A1c Analysis System.

FIG. 8 shows the cross section of a Hemolysis Module where optical fibers, LED and photo detector components (not shown) may be embedded to measure Optical Density of the hemolysate.

FIG. 9 shows the static mixer element and means for reciprocal mixing.

FIG. 10 shows a photograph of a micropump for providing means to deliver reagent through the fluidics system.

FIG. 11 shows an integrated mobile phase pre-heater (A) and a cartridge oven (B).

FIG. 12 shows a cross section view of the chromatographic cartridge and connectors.

FIG. 13 is a graph presenting chromatographic data that has been acquired from a sample that contains glycated A1c.

FIG. 14 is a graph of chromatographic data taken from plasma proteins at a selected concentration.

FIG. 15 is a graph of chromatographic data taken from plasma proteins at a selected concentration.

FIGS. 16, 17, 18, 19, 20 and 21 are graphs comparing data that has been acquired from a sample or specimen to a comparison baseline to provide a sample analysis.

### DETAILED DESCRIPTION

The system disclosed herein may include a plurality of modules to accomplish processing and analysis of blood samples. A module may include a single element or a plurality of elements designed to operate in a concerted manner to achieve a system functionality. An element may be a device or component that functions as a stand alone unit. Alternatively, an element may contain two or more devices accomplishing a common function. For purpose of this disclosure, the substance of interest may be a compound, a protein, a nucleic acid or their derivatives. Examples of a compound may be a small organic molecule, an amino acid and its derivative or a small peptide. The sample is preferably in a liquid form, and is most preferably a blood or plasma sample.

The samples to be quantitated may be held in a container. In a preferred embodiment, at least one end section of the container may be fit between the arms of the gripper when the arms are open to the fullest extent. Most preferably, the cross section of at least one end section of the container has an oval or circular shape, such as a tube, so that the container may rotate after the gripper arms are closed.

A multi-fingered tube gripper may be used to translocate the sample containers from the rack to the system. A scannable bar code is preferably affixed to the outside of each container before the sample containers are presented to the system. In one preferred embodiment, the gripper permits the container to rotate around the container's vertical axis to enable sample identification via a bar code reader.

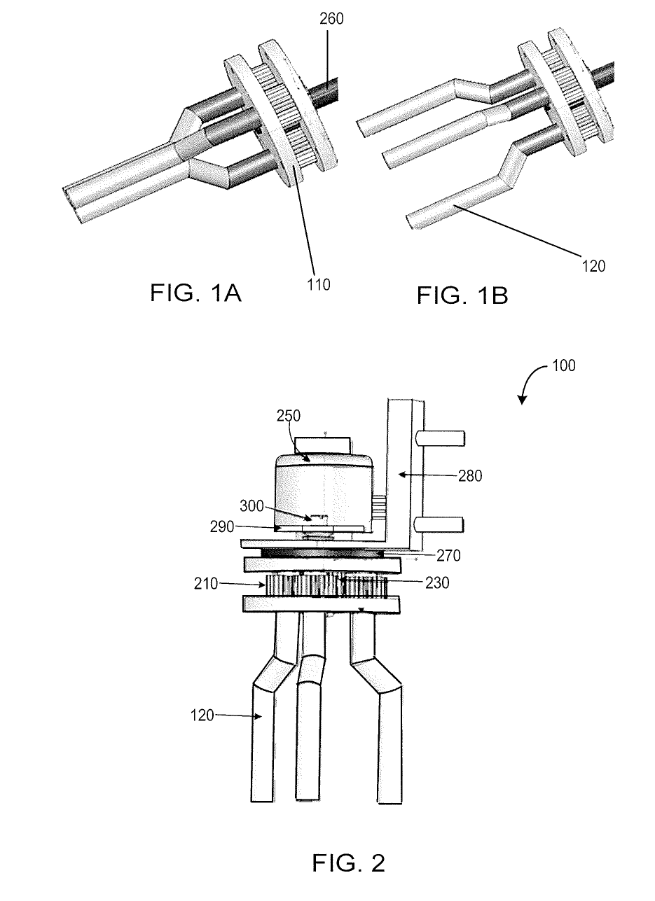

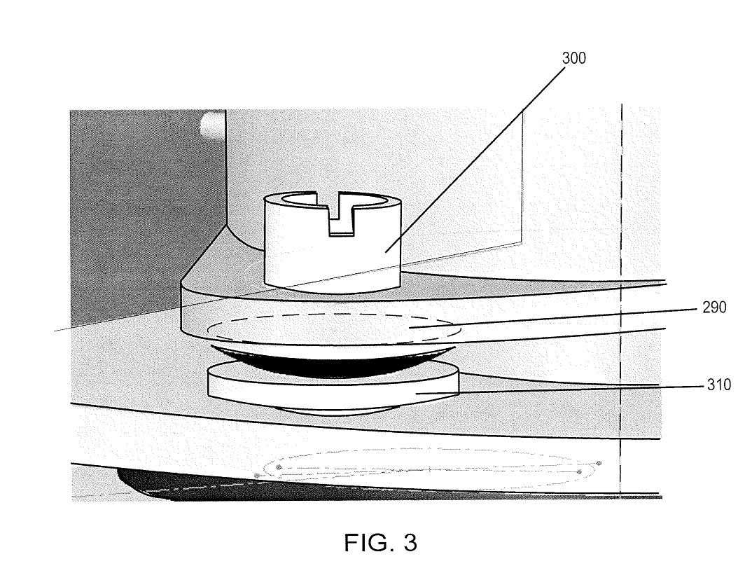

As shown in FIG. 1, the design of the gripper module 100 employs a housing unit 110 containing multiple gears and three or more gripper arms 120. The gripper arms 120 may open (FIG. 1B) and close (FIG. 1A) as driven by the gears in the housing 110. The gripper arms 120 contact each other when they close to a minimum radius (FIG. 1A), and the arms may be open to the maximum when a container is to be released. Intermediate positions also exist when the gripper arms 120 grip on the outer wall of a container. For purpose of this disclosure, these intermediate positions where the arms are open to an extent that is between the “fully open” (FIG. 1B) and “fully closed” (FIG. 1A) positions are referred to as “closed-on-tube.” Under a closed-on-tube situation, the opening radius of the gripper arms 120 varies depending on the size of the sample container.

FIG. 2 illustrates the design of the gripper in greater details. Three or more spur gears 210 are connected to offset shafts (or “gripper arms”) 120 and are symmetrically located around a driver gear 230 that is attached to a single reversible motor 250. The motor 250 may be under the control of an electronic controller (not shown). The motor output shaft 260 (as shown in FIG. 1) protrudes through the housing 110 and is connected to the driver gear 230.

The series of three or more offset gripper arms 120 with spur gears 210 are concentrically constrained in a two-part circular cup-shaped housing 110 in contact with a central driving gear 230 connected to the output shaft of the motor 250. A friction disk 270 between the top of the gear housing assembly 110 and the gripper mounting bracket (or flange) 280 and the motor mounting bracket (or flange) 290 constrains the motion of the housing 110 until the motion of the gripper arms 120 either lock the gear motion at the center (i.e., at fully closed position) or come into contact with the top of the tube located in their field of motion (i.e., closed-on-tube).

When the offset shafts 120 are at the closed-on-tube position or at the fully closed position, the static friction of the friction disk 270 may be overcome and the housing 110 begins to rotate. This motion provides a means to present a bar code (not shown) to a barcode reader without the need for a separate motor to independently rotate the tube about its vertical axis. Sample containers may be released by reversing the rotation of the motor when they have been placed securely at their destination. Thus, both the translocation and rotation of sample containers may be accomplished by the same motor 250.

Force to grip the sample containers may be adjusted by tightening two inverted T-nuts 300. The T-nuts are preferably threaded pop-rivets nuts on stud screw fastened to the motor mounting bracket 290 to compress bell-washer (or bell spring) 310 under the motor mounting bracket 290.

The rotation of the housing may be achieved by incorporating a series of small magnets placed concentrically around the perimeter of the spur gear housing and use of a Hall Effect sensor to detect rotation by changes in magnet field strength. Alternatively, the rotation of the housing may be confirmed by use of a circular optical encoder disk with sensor feedback.

The status of the gripper arms in either open, closed or intermediate positions may be determined by an optical sensor similar to that employed in a computer mouse or an optical encoder reading a radially encoded pattern centered on the top of one of the offset gripper arms. Gripper arm positions may also be determined by a magnetic field sensor positioned above a small magnet whose magnetic axis is orthogonal to the long axis of the offset gripper arm.

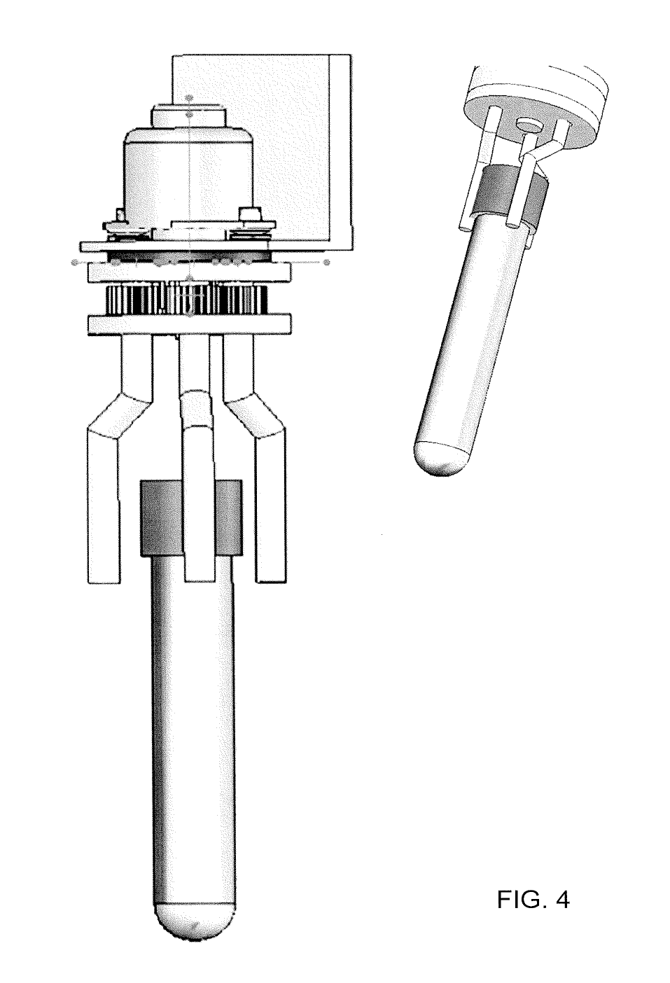

Before being presented to the system, the containers (or tubes) may sit in a multi-sample tray. When the gripper arms are positioned around the axis of a tube (FIG. 4A), the arms may be closed by action of the motor and the tube is gripped (FIG. 4B). After the tube is placed in the receiving station of a receiving device, the gripper arms are opened and the tube is released by the rotation of the motor.

A receiving device may be designed to hold a single tube or multiple tubes presented by the gripper module 100. In one embodiment, the receiving device is designed such that it may receive and resuspend (mix) sedimented red blood cells to present a uniform sample of whole blood to the hemolysis and sample analysis modules. After analysis, the tubes may be presented to the gripper assembly in a similar manner for re-identification before returning to the sample tray.

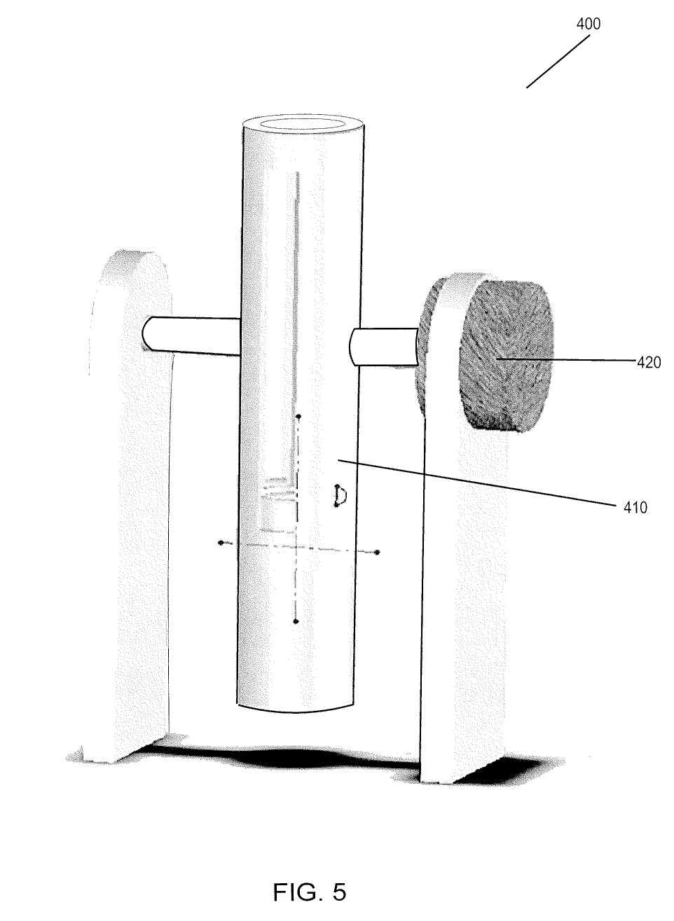

FIG. 5 shows a single tube receiving device and mixer 400. A tube holder 410 holds the sample container (not shown), and a motor 420 drives the movement of the tube holder. Any movement ranging from vertical through horizontal motion of the tube may be employed to mix the blood cells. After mixing, the tubes may be presented to the sampling needle so that blood samples may be withdrawn for subsequent presentation to the blood processing modules.

The fluidics flow scheme and multi-function valves of the blood processing modules are illustrated as shown in FIG. 6. The sample container from the mixer may be presented to the syringe piercing septum sampling needle. Whole blood flows may be driven by mechanical displacement which may be generated by a pumping device, such as a syringe pump. Blood samples may be input into one of the chambers in a valve rotor. The valve rotor preferably has multiple chambers. In the most preferred embodiment, the number of chambers is 6. The rotation of the valve rotor may transport the blood from one chamber to another. Some chambers may be connected to different blood processing modules, while some chambers may not be connected to any blood processing modules. Thus, the rotation of the valve motor may render the blood sample accessible to different blood processing modules, which may include a sample intake module, a hemolysis module, a separation module containing a column or a disposable cartridge. Those chambers that are not connected to the blood processing modules may serve as intermediate stops. In a preferred embodiment, certain chambers that are not connected to the blood processing modules are connected to a rinsing device. This prevents cross-sample contamination.

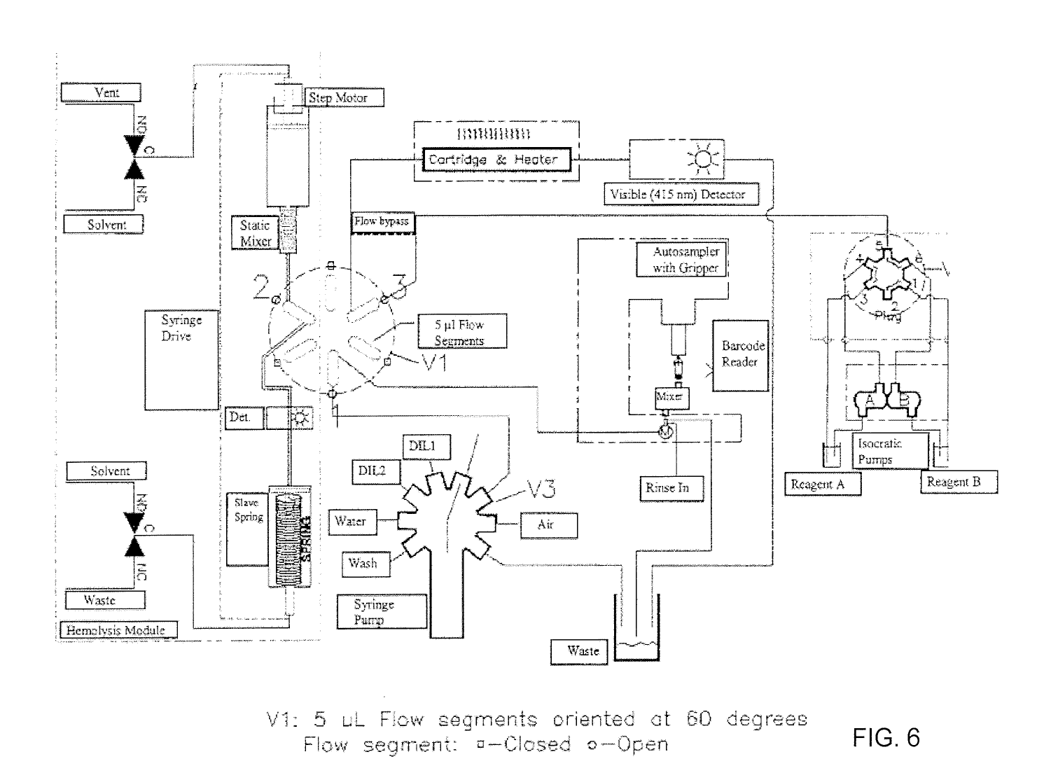

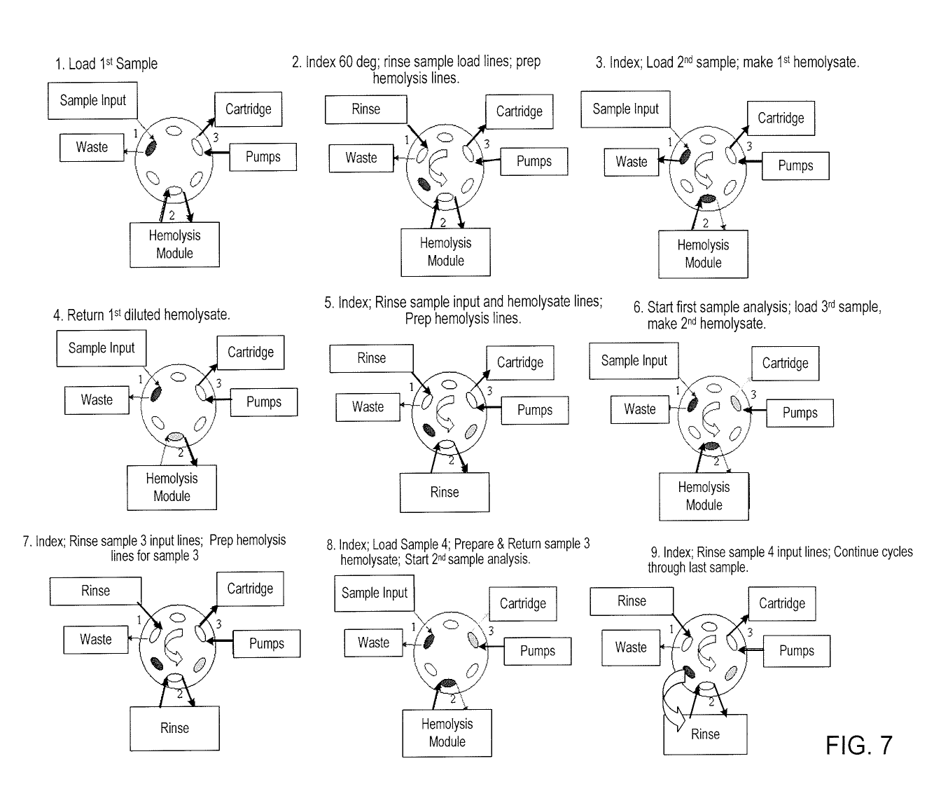

By way of example, the sample pipeline process is outlined in the following text and illustrated in FIG. 7. Three valve rotors, V1, V2 and V3, are shown in FIG. 6 for purpose of illustration but not limitation. In Step 1, blood samples may be input from the sample receiving and mixing module through a port (e.g. V1-Port 1 in FIG. 6) into one of the chambers in a valve rotor. Subsequent indexing of the valve 60 degrees in Step 2 may serve to isolate the 5 μL sample from the fluidics (V1-position between Ports 1 and 2). This motion presents new slots to Ports 1, 2 and 3 for flow continuity and enables rinsing of Port 1 (the blood sampling flow path) prior to receiving a new, whole blood sample.

A second sample may be loaded in Step 3 after indexing the valve 60 degrees, while the first sample may be presented to the hemolysis module. To facilitate lysing, the hemolysis module may have its lines filled with diluents prior to receiving the blood sample. Step 4 returns the hemolysate to the chamber while the valve rotor V1 remains static.

Indexing the valve in Step 5 may isolate the second blood sample and the hemolysate of the first sample while the sample intake and hemolysis module lines are rinsed and pre-filled with diluents. Indexing the valve rotor in Step 6 may present the hemolysate of the first sample to the separation module for analysis. In the meantime, the second blood sample may be presented to the hemolysate module while a third blood sample may be taken in by the sample intake module. Further rinsing of the injection Port 3 o is not required because of the continuous flow from the pumps.

In Step 7, the valve rotor V1 may remain static while the hemolysate of the second sample is returned and the cartridge of the separation module is continuously rinsed. Subsequent indexing the valve rotor may isolate the third blood sample and the hemolysate of the second sample. In Step 8, V1 is indexed and second sample may be loaded to the column, the third blood sample may be lysed and the sample intake may take in a fourth sample.

Indexing of V1 in Step 9 may allow the return of the hemolysate of the third blood sample and continue rinsing of the column and the sample intake and hemolysis lines.

In essence, Steps 1-8 may represent the sequence of steps an individual sample may go through in the blood processing modules. Steps 1-8 may be repeated in this sequence for pipe-lining blood samples for analysis by the disclosed system.

Prior to presenting a blood sample to the hemolysis module, the inlet fluidic segment may be preloaded with the first portion of diluents (about 125 μL). This may facilitate subsequent red blood cell rupture. When the valve rotor V1 is indexed 60 degrees from the intermediate position to present the blood sample, an additional amount of diluents (about 100 μL) may be used to displace the 5 μL of blood sample into the hemolysate module. More diluents may then be added as desired.

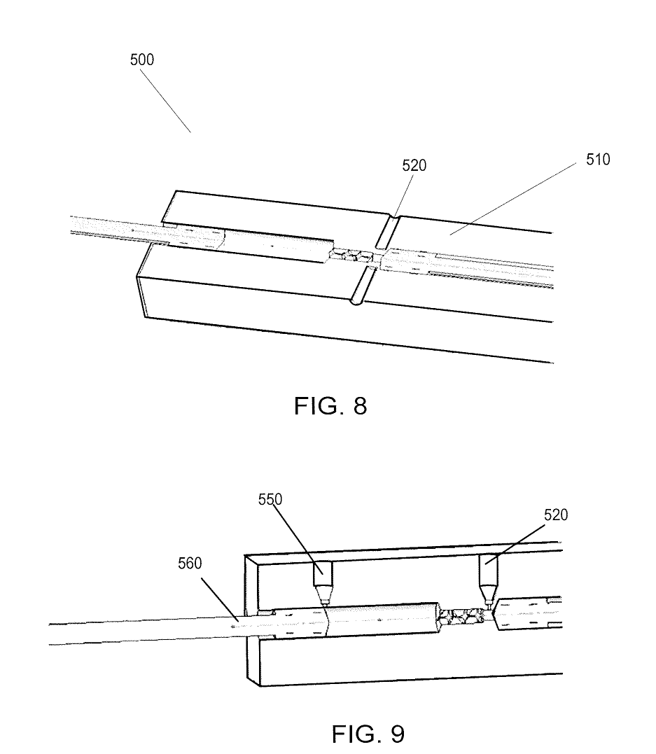

The blood sample may arrive at Port 2 by means of valve rotor indexing and may be transferred to one of the two mixing chambers of the hemolysis module (FIG. 6, area bounded by dotted lines) during Step 3 by means of diluents displacement. Thereafter, the open flow path between the two mixing chambers allows rapid transport of fluid from the chamber receiving the hemolysis fluid through the static mixer and into the transparent section providing an integrated cuvette and onto the second chamber (FIG. 8). Turbulent mixing may occur when the liquid is repeatedly transferred between the two chambers. This may facilitate hemolysis of the red blood cells.

The Hemolysis Module body may include a static mixer 500 which may be machined from optically clear acrylic to form a mixing block 510 with optical view ports 520 centered on liquid entry adjacent to static mixer (not shown). A vent 550 may be designed to be present at the left top port to allow liquid entry through the center port. The static mixer is preferably placed in an optically dark housing to avoid stray light.

Optical fibers, LED and photo detector (not shown) may be embedded in the static mixer to measure Optical Density of the hemolysate. After mixing, by transfer between the opposed chambers (FIG. 9), the concentration of hemoglobin may be determined by measuring the light absorption (O.D.) of the mixed solution using a LED-based detector. The light path of the detector is preferably orthogonal to the view shown in FIG. 8. The concentration of Hemoglobin derived from the hemolysis of red blood cells may be determined by measuring the diluted sample's Optical Density using absorbance at 415 nM wavelength. If necessary, additional diluents may be added with remixing to achieve the desired concentration of hemoglobin prior to returning the hemolysate to the rotor.

The static mixer may include two pistons 560. The left piston is positioned such that flow (containing 5 μL blood and 195 μL transfer rinse) may enter the left most chamber flowing through the static mixer and after deliver of the fluid, the two pistons may move in a synchronous manner to the right and left. To achieve better mixing results, the second left motion may be constrained such that the fluid is not exposed to the vent port 550. Because the view ports 520 are located in the center of the liquid flow, absorption of light can be used to calculate the hemoglobin concentration and additional dilution may be made if desired.

The piston shaft protruding from the block may be sized to accommodate a 10-32 thread that may be mated to a motor. This may provide the rapid motion required for turbulent or shear flow through the static mixer. Periodic treatment of the chamber with a proteolytic enzyme may help prevent build up of cellular debris and a rinse solution containing alcohol and Triton-X100 surfactant may be used to minimize accumulation of lipid moieties.

The hemolysis sample may be returned from the hemolysis chamber to the valve rotor V1. In one embodiment, 5 μL of hemolysate may be captured upon a 60-degree rotation of the valve and deposited in the appropriate chamber of the rotor (FIG. 6, Port 2). Following rinsing of the flow path of the hemolysis module, the hemolysate sample may be immediately transferred to the injection port after being temporarily held in the intermediate position between Port 2 and Port 3 (FIG. 6). The rotor slot following the rotor's intermediate blocked flow or pause position allows the flow path of the hemolysis module to be rinsed before receiving the next sample.

Two micro-processor controlled, stepper-motor based precision reagent pumps may be used to drive the fluidics through the chromatographic column. The two pumps may provide for overlapped fill/delivery of Reagent A (retention buffer or Buffer A) or Reagent B (release Buffer, or Buffer B) at flow rates of up to 2.5 mL/min and delivery pressure of about 200-300 psi. The analysis cycle may be set at 60 seconds or less for each sample.

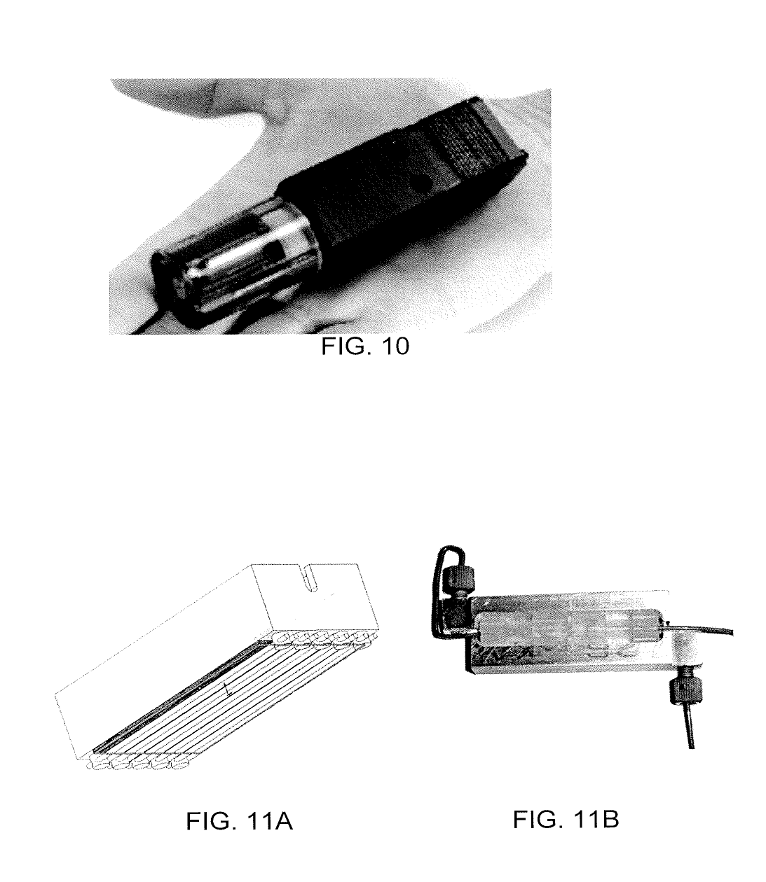

In one embodiment, micropumps manufactured by Sapphire Engineering (FIG. 10) which incorporate microprocessor controlled motors may be used to provide a means for delivering reagent through the analytical fluidics system including but are not limited to the injector valve, the mobile phase pre-heater, the separation cartridge and the detector.

A Mobile phase Pre-heater (FIG. 11A) and Cartridge Oven (FIG. 11B) may be integrated into the system to maintain the temperature of the mobile phase and Cartridge at about 55 C. The Pre-heater may be attached to the back of an Aluminum block with pressure sensitive adhesive and a 15 cm of 1/32″ ss tubing may be held against the heater with Aluminum foil adhesive-backed tape. The tubing is attached to the 1/16×0.010″ i.d. secured by Upchurch's 10-32 NF finger-tight fittings.

A Minco CR-198 controller and self-regulated foil-backed Capton heater may be used to provide Temperature control. The heater may be fastened by pressure sensitive adhesive. Contact with the 1/32″ ss pre-heater tubing is ensured by self adhesive Aluminum tape which renders the tubing captive to the heater. Connection of the 1/32″) od ss. pre-heater tubing may be effected by two flat faced ferrules captive in the machined port by a ¼″ 28 NTF PEEK male connector seated in opposite ends of the cartridge heater (FIG. 11B).

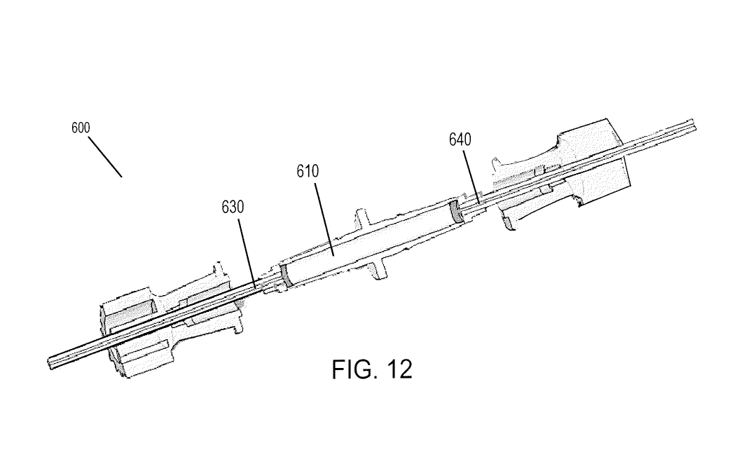

Cartridges 600 of the separation module may be derived from a Luer male-to-male adapter fitting 610 into which a 0.125″ dia.×0.040″ thick Teflon, stainless steel or a Titanium 5 micron frit 630 is press-fit (FIG. 12). A second frit 640 may be press-fit into the top of the cartridge after it is filled with 20 micron HEMA-based resin particles that have been reacted to obtain surface-linked with a phenylboronate entity. Two Luer adapter fittings may be employed to connect the column cartridge to the 1/16″×0.010″ id PEEK reagent tubing. Two 1/16″×0.010″ id PEEK reagent tubes connect the cartridge via flange seal fittings to complete the fluidic path. Two Luer retainer rings that lock onto the Luer cartridge may be used to engage the Luer adapter fittings (not shown).

A mobile phase suitable for the binding of the proteins to the column is selected and may contain an acetate buffer pH 8.0 or greater. The module may also include a mobile phase pre-heater and a cartridge containing a chemically attached aminophenyl boronate which is suitable for the binding of the proteins of interest. Porosity of the boronated column may be from about 100 A (Angstrom) to about 1000 A. For example, 100 A or 300 A in pore size is generally adequate for the binding of hemoglobin, glycated plasma proteins, or in the case of larger proteins, 1000 A is adequate.

Particle size for boronated column packing material may range from 20 microns to 200 microns in diameter. This allows for the advantageous operational pressure of about 13.8 bar or 200 pounds per square inch (psi) or less at flow rates between 5 and 100 micro liters per minute. This larger particle size and lower operating pressure offer a significant advantage because low pressure pumps and related components rated for low pressure operation may be used in place of the high pressure pumps and related components rated for high pressure operation, such as those described in U.S. Pat. No. 6,020,203. The lower pressure liquid chromatography system may reduce costs associated with hardware, operating and maintenance when compared to HPLC (high pressure liquid chromatography), which is used by other systems.

The cartridge heater may be integrated into the system to maintain the temperature of the mobile phase and Cartridge at about 55° C. The Pre-heater may be attached to the back of an Aluminum block with pressure sensitive adhesive and 15 cm of 1/32″ stainless steel tubing may be held against the heater with Aluminum foil adhesive-backed tape. The tubing is attached to the 1/16″×0.010″ i.d. secured by such means as Upchurch's 10-32 NF finger-tight fittings. In another implementation, the pre-heating function may be performed in other portions of the fluidics module, such as in special valves or tubing or the like, transited prior to presentation of the sample to the column.

A visible spectrum detector may be used to monitor the optical density of the effluent of the cartridge at 415 nM. A computer program may be used to record the chromatographic profile after the absorbance values of the detector's analogue output is digitized at approximately 20 pts/sec. The program may provide data reduction to yield chromatographic baseline corrected area ratios for patient samples; these ratios may be subsequently converted to A1c values via the system's current calibration curve. This curve may be derived from the analysis of certified samples of known A1c value. Control charts for data quality may be obtained by plotting analytical results for control samples against the range of their known values.

### EXAMPLES

The following examples illustrate the operation of the system disclosed herein. The examples set forth in this disclosure are illustrative, not exhaustive. The materials, chemicals and other ingredients are presented as typical components or reactants, and the procedures described herein may represent but one of the typical ways to accomplish the goals of the particular procedure. It is understood that various modifications may be derived in view of the foregoing disclosure without departing from the spirit of the present disclosure.

### Example 1

The analysis is accomplished by diluting a blood or serum or plasma or similar sample in a diluent such as DI water or buffer commonly known to those experienced in the art, injecting such sample into a chromatography system, and monitoring the effluent at an appropriately chosen wavelength.

Specifically in the case of A1c, the binding buffer is pumped through the system first, causing the glycated species to bind to the column. Porosity of the boronated column is 100 A or thereabouts. Such binding buffer is chosen from the likes of acetate or phosphate, carbonate, or such similar buffer in concentrations of 0.01 to 0.4 M concentrations range and a pH in the range of 6 to 11. The buffer is pumped through the system until the non-glycated portion is eluted. Following this, the glycated portion of the protein is eluted by switching the buffer to a new buffer consisting of an elution component which may have an acidic pH, or containing a more competitive glycol additive such as mannitol.

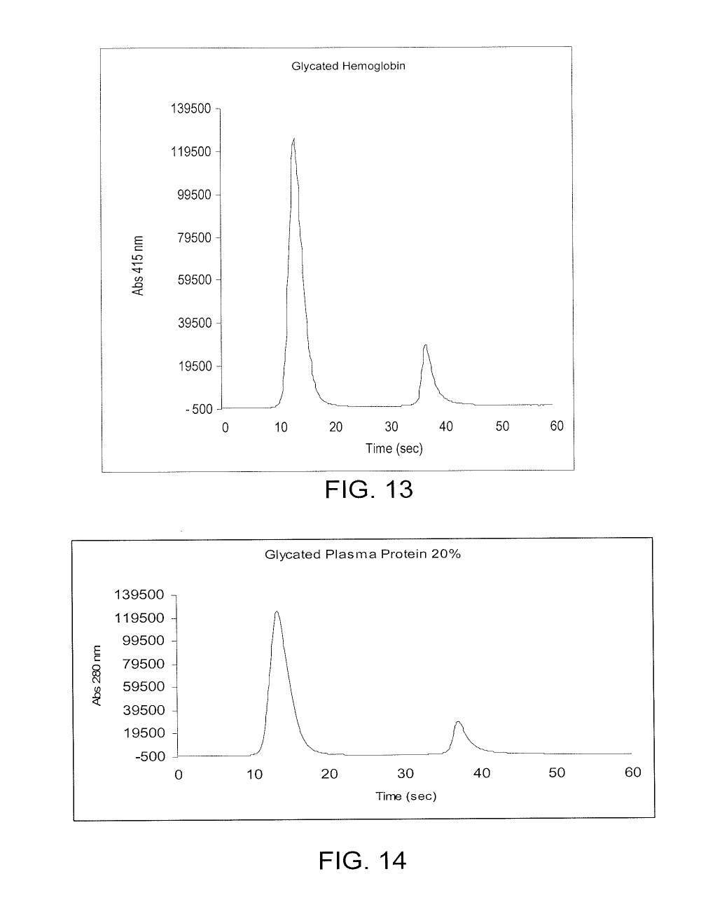

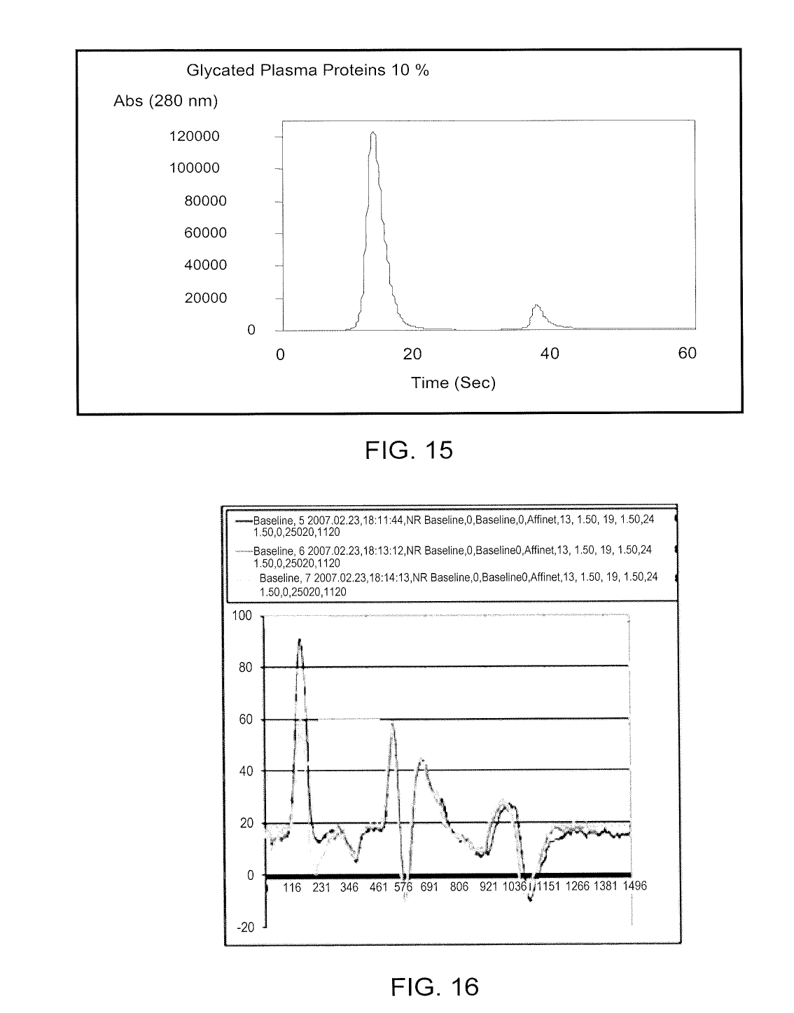

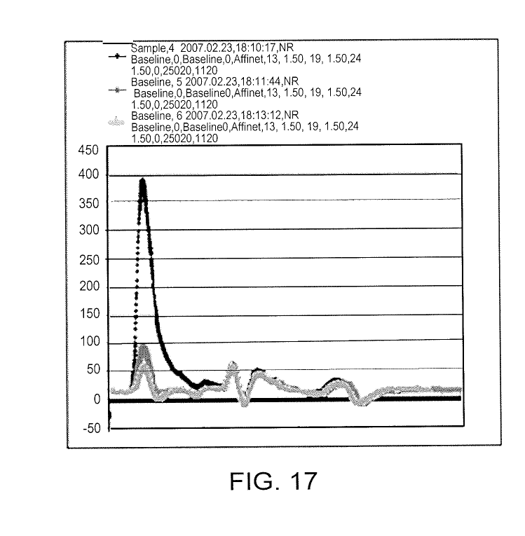

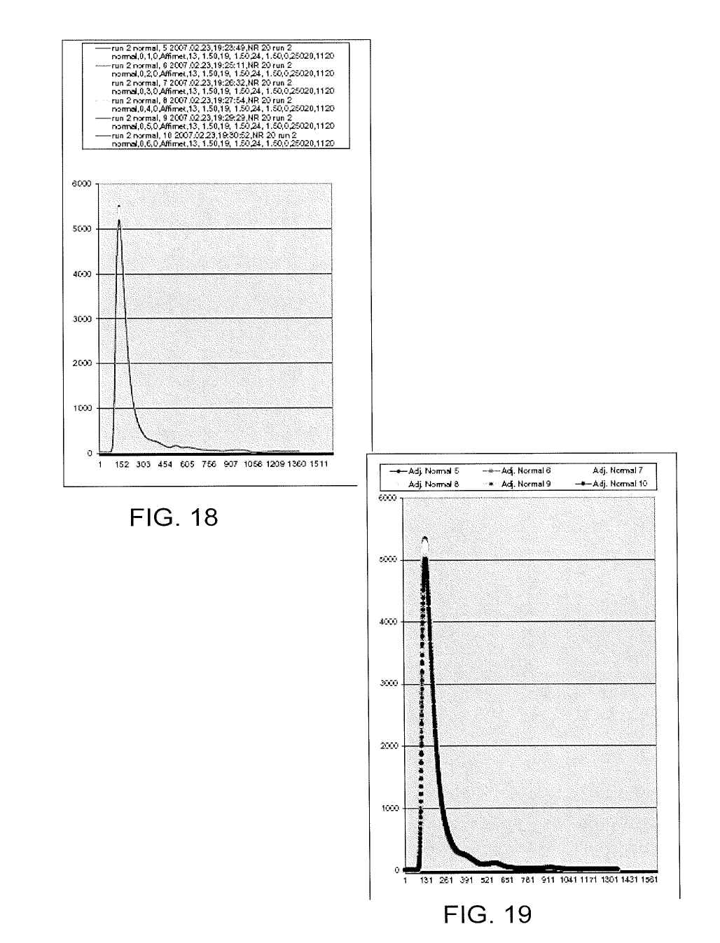

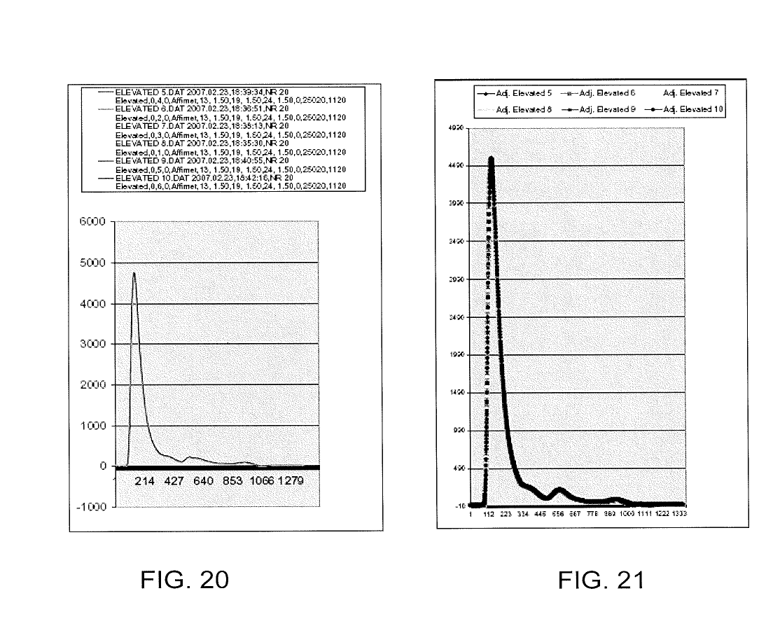

In the case of glycated A1c, such a system may yield chromatographic presentations similar to the elution curve shown in FIG. 13. When plasma proteins are analyzed, a profile similar to that of FIG. 14 (for 20% glycated plasma protein) or FIG. 15 (for 10% glycated plasma protein) is observed. FIGS. 16-21 show what may be done in an analytical mode to compare data that is acquired from a sample to a baseline of data from a known or control sample.

Analytical conditions that may be used to accomplish the separations include Buffer A, 0.5 to 2.5 mL/min for 10 to 30 sec, then Buffer B at 0.5 to 2.5 mL/min for 10-30 sec, followed by buffer A at 0.5-2.5 mL/min. The reproducibility of the analysis is typically <5% CV at 10.0% A1c after calibration.

Other patents, patent applications and other literature have been cited throughout this disclosure. The contents of these citations are hereby expressly incorporated into this disclosure by reference.

We claim:

1. A system for quantitating a substance of interest in a sample, comprising:
(a) a chromatography module for analysis and/or purification of the substance of interest;
(b) a detector module for sensing a quantifiable parameter of the substance of interest, and producing a signal representative of the quantifiable parameter; and
(c) means for resolving the signal to quantify the quantifiable parameter as a quantification result, and for reporting the quantification result.

2. The system of claim 1, further comprising means for quantitating plasma proteins that are separated from their glycated analogs.

3. The system of claim 1, further comprising the sample, wherein the sample is a blood sample.

4. The system of claim 3, wherein the substance of interest is hemoglobin A1c.

5. The system of claim 1, wherein the chromatography module comprises a disposable chromatographic column.

6. A system for quantitating a substance of interest in a sample, comprising:
(a) a chromatography module for analysis and/or purification of the substance of interest, wherein the chromatography module comprises a disposable chromatographic column that contains boronated packing particles of from 20 to 200 microns in diameter, said column being capable of separating the substance of interest at a flow rate from 5 to 100 micro liters per minute and an operation pressure of 200 psi or less.
(b) a detector module for sensing a quantifiable parameter of the substance of interest, and producing a signal representative of the quantifiable parameter; and
(c) means for resolving the signal to quantify the quantifiable parameter as a quantification result, and for reporting the quantification result.

7. A system for quantitating a substance of interest in a sample, comprising:
(a) a gripper module that translocates containers holding the sample to be measured;
(b) a mixing module for mixing the samples in respective containers;
(c) a chromatography module for purification of the substance of interest;
(d) a detector module for sensing a quantifiable parameter of the substance of interest, and producing a signal representative of the quantifiable parameter; and
(e) means for resolving the signal to quantify the quantifiable parameter as a quantification result, and for reporting the quantification result.

8. The system of claim 7, further comprising a bar code reader to obtain the bar code information from the individual containers.

9. The system of claim 8, further comprising a hemolysis module for lysing the red blood cells in the sample.

10. The system of claim 7, wherein the substance of interest is hemoglobin A1c.

11. The system of claim 7, wherein the a chromatography module comprises a disposable chromatographic column.

12. A system for measuring glycated hemoglobin A1c, comprising:
(a) a gripper module that translocates containers holding the sample to be measured;
(b) a mixing module for mixing the sample in the container;
(c) a chromatography module for purification of the substance of interest, said chromatography module comprising a disposable chromatographic column; and
(d) a detector module for measuring the optical density of the substance of interest.

13. A disposable chromatographic column comprising
(a) a column body suitable for packing with materials ordinarily used in the industry and which is used in the practice of liquid chromatography, sample preparation, purification and the like;
(b) means for retaining the column packing materials by insertion of retaining frits on either end of the column body;
(c) means for connecting the column body to a chromatography system by the use of simple Luer type fittings; and
(d) means for storing the columns through the use of Luer end caps to prevent the column packing materials from drying out.

14. A method for quantitating a substance of interest, comprising
adjusting the concentration of said substance to fall within a narrow, specified range of values; and
calculating the original concentration of the substance by retrograde conversion.

15. The method of claim 14, wherein the substance of interest is hemoglobin A1c.

16. A method for analyzing a liquid sample each containing both glycated and non-glycated protein fractions, comprising the steps of:
(a) providing an affinity chromatographic column;
(b) contacting said column with said liquid sample containing said glycated and non-glycated protein fractions,
(c) separating said non-glycated protein fraction from said glycated protein fraction of the liquid sample with said column at an operation pressure of 200 psi or less.

17. The method of claim 16, wherein said column is a disposable chromatographic column.

18. The method of claim 16, further comprising the step of identifying each liquid sample through a bar code pre-affixed to the container holding said sample.
